(19) United States
(12) Patent Application Publication
(10) Pub. No.: US 2009/0254062 A1
(43) Pub. Date: Oct. 8, 2009

(54) Infusion catheters with slit valves and of simplified construction

(76) Inventors: Mark W. McGlothlin, San Diego, CA (US); Scott W. Herrick, San Diego, CA (US)

Correspondence Address: STAHL LAW FIRM, 2 MEADOWSWEET LANE, SAN CARLOS, CA, 94070, US

(21) Appl. No.: 12/080,572

(22) Filed: Apr. 3, 2008

(51) Int. Cl. A61M 25/00; B26D 3/00

(52) U.S. Cl. 604/508; 604/523; 83/13

(57) ABSTRACT

The present invention relates to catheters to deliver a fluid to a patient. A catheter of the invention comprises a plurality of slits through which the fluid is delivered to the patent. A catheter of the invention is designed in a simple manner to lower costs and provide a reliable means to deliver fluids to a patient. The invention further comprises methods for making a catheter of the invention and methods of delivering a fluid to a patient with a catheter of the invention.

### 1.0 FIELD OF THE INVENTION

The present invention relates to catheters for medical uses and methods of making and using such catheters.

### 2.0 BACKGROUND

Catheters are used to deliver fluids into a patient, including humans and animals. The overall shape of a catheter is typically that of a thin tube which can be placed into the blood stream of a patient. Once placed into a patient, a catheter releases a fluid into the patient. Many medical fluids are preferably delivered to a patient at a certain rate so the patient will receive fluid, for example a drug containing fluid, at a desired rate and therefore the drug at a desired dosage. Ensuring a desirable delivery of the fluid to the patient is a challenge in the design of catheters. A catheter should be able to deliver a fluid over an extended period of time, preferably with a flow rate that is sufficiently workable to address the therapeutic needs of the patient. A catheter should be able to deliver a fluid over a segment of its tube-like shape (infusion segment) to distribute the fluid in the patient and to avoid very high flow rates and pressures at one location. When delivering a fluid over an extended segment along a catheter, it can be a challenge to ensure that the flow rate is similar or even about the same along the infusion segment, and that it remains so over the duration of the infusion period.

Catheters are preferably used in a disposable fashion. Also, catheters are often used in considerable numbers. The cost of applying a fluid to a patient can be considerably affected by the cost involved in the making of a catheter. A catheter design that facilitates an effective delivery of medical fluids while being affordable would be highly desirable.

Catheter designs have attempted to address some of these concerns. However, a catheter that can better deliver a fluid to a patient while having a cost-saving design would be highly desirable. The present invention provides such catheters and methods of making and using such catheters.

### 3.0 SUMMARY OF THE INVENTION

The present invention relates to catheters and methods of making and using such catheters. A catheter of the invention, in certain embodiments, comprises an inner lumen through which fluid flows into the catheter for delivery to a patient. In certain other embodiments, a catheter of the invention comprises two inner lumens through which a fluid may flow for delivery to a patient, or three, or four inner lumens. An inner lumen of a catheter of the invention, in certain embodiments, may have a cross-section in a shape that facilitates flow and delivery of fluids to a patient, most preferably round.

A catheter of the current invention, in certain preferred embodiments, comprises slits through which a fluid can be delivered to a patient. In most preferred embodiments, a catheter of the invention comprises multiple slits, preferably in the distal portion of the catheter.

In certain other preferred embodiments, a catheter of the invention can be utilized to achieve uniform or substantially uniform distribution of fluid over the distal end of the catheter, preferably at low flow rates and low pressures.

The invention further comprises methods for delivering a fluid to a patient, including a human and an animal. The invention also relates to methods for making a catheter of the invention.

### 4.0 BRIEF DESCRIPTION OF THE DRAWINGS

FIG. 1 is an example of a catheter of the current invention (1) with an infusion segment (2). The exemplified catheter is connected to an infusion pump connector (3), a flow restrictive element (4), a flow restrictor connector (5), a filter (6), and an elastomeric infusion pump (7) with a liquid fill port (8).

FIG. 2 shows examples of cross sections of catheters of the invention. Exemplified are a single-lumen catheter (A and B) and a dual-lumen catheter (C). Illustrated are the lumen (9) of the catheter and the delivery slit (10).

FIG. 3 is an example of delivery slits (10) in an infusion segment (11) of a catheter of the invention with a printed band at the proximal end of the infusion segment (12) and a closed distal end (13).

FIG. 4 is an example of delivery slits (10) arranged in a spiral in an infusion segment (11) of a catheter of the invention with a printed band at the proximal end of the infusion segment (12) and a closed distal end (13). The dotted lines in the infusion segment (11) represent delivery slits (10) that are located on the opposite site of the catheter.

FIG. 5 is an example of delivery slits (10) arranged in an angled manner in an infusion segment (11) of a catheter of the invention with a printed band at the proximal end of the infusion segment (12) and a closed distal end (13).

FIG. 6 is an example of delivery slits (10) shorter than those exemplified in FIG. 3 in an infusion segment (11) of a catheter of the invention with a printed band at the proximal end of the infusion segment (12) and a closed distal end (13).

FIG. 7 is an example of delivery slits (10) longer than those exemplified in FIG. 3 in an infusion segment (11) of a catheter of the invention with a printed band at the proximal end of the infusion segment (12) and a closed distal end (13).

FIG. 8 is an example of delivery slits (10) that are spaced more closely than those exemplified in FIG. 3 in an infusion segment (11) of a catheter of the invention with a printed band at the proximal end of the infusion segment (12) and a closed distal end (13).

FIG. 9 is an example of delivery slits (10) that are spaced further apart than those exemplified in FIG. 3 in an infusion segment (11) of a catheter of the invention with a printed band at the proximal end of the infusion segment (12) and a closed distal end (13).

FIG. 10 is an example of delivery slits (10) in two infusion segments (11) of a catheter of the invention with dual lumen. Also exemplified are flow restrictive channels (14), a high pressure fluid manifold (15), and an infusion set tubing (16).

FIG. 11 exemplifies a method for making a delivery slit in a catheter of the invention by piercing with a lancet (17) through the catheter wall (18).

### 5.0 DETAILED DESCRIPTION OF THE INVENTION

The present invention relates to catheters capable of delivering a fluid at a desirable rate. Catheters of the invention, in certain embodiments, are capable of delivering a fluid over an infusion segment along the catheter. In certain other embodiments, a catheter of the invention is designed in a simple manner to lower manufacturing costs.

In certain other embodiments, the invention comprises methods for making a catheter, preferably a method for making a catheter of the invention. In certain other embodiments, the invention comprises methods for using a catheter of the invention.

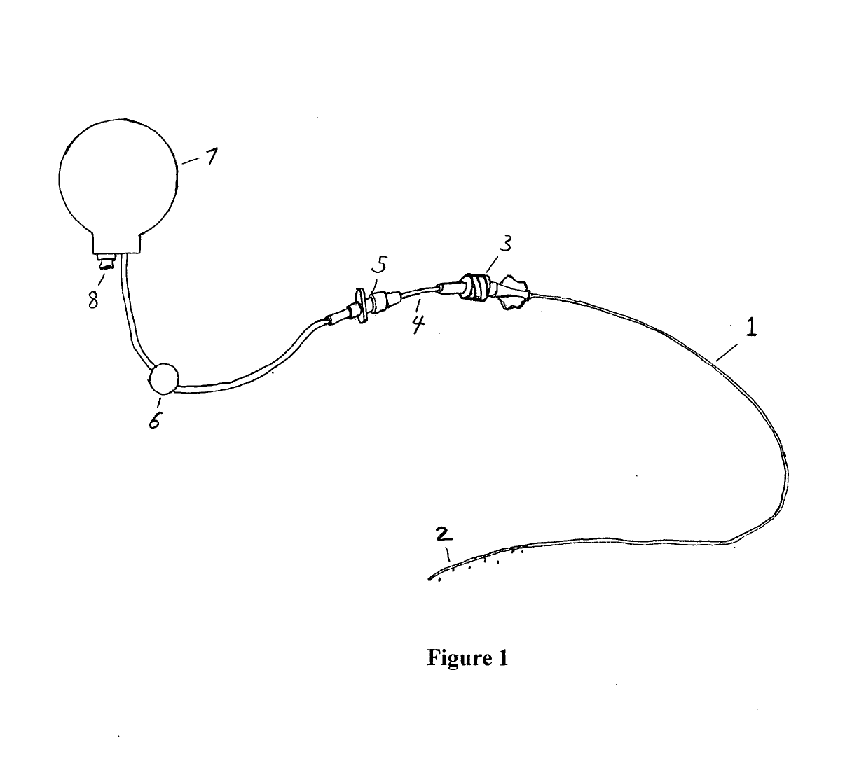

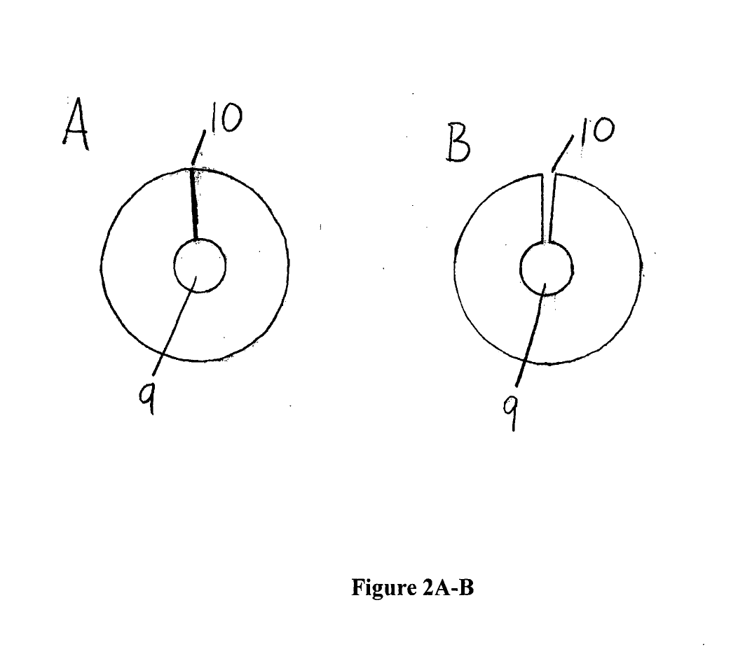

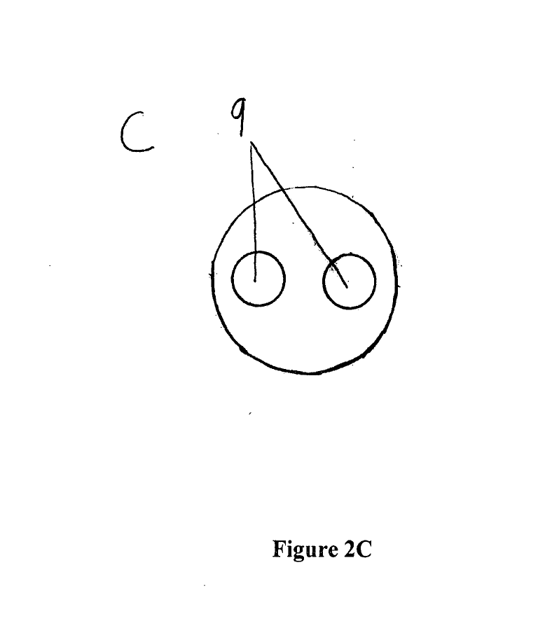

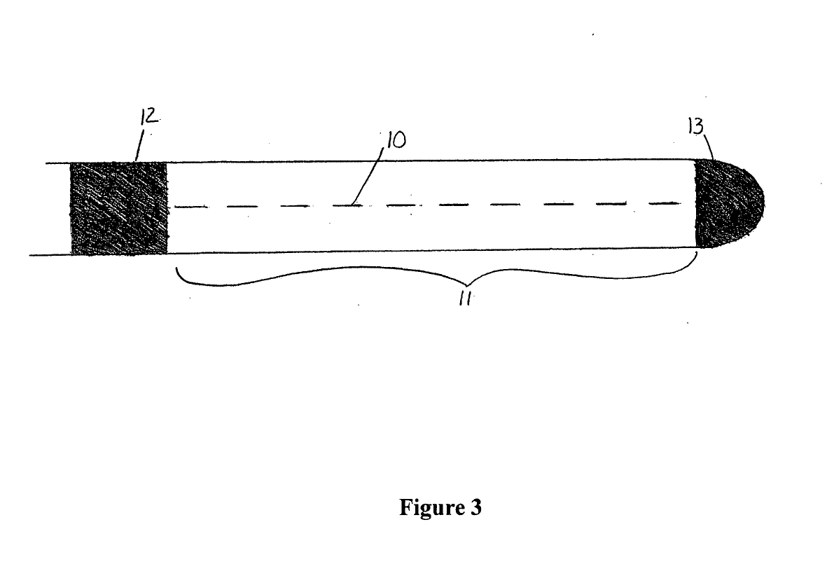

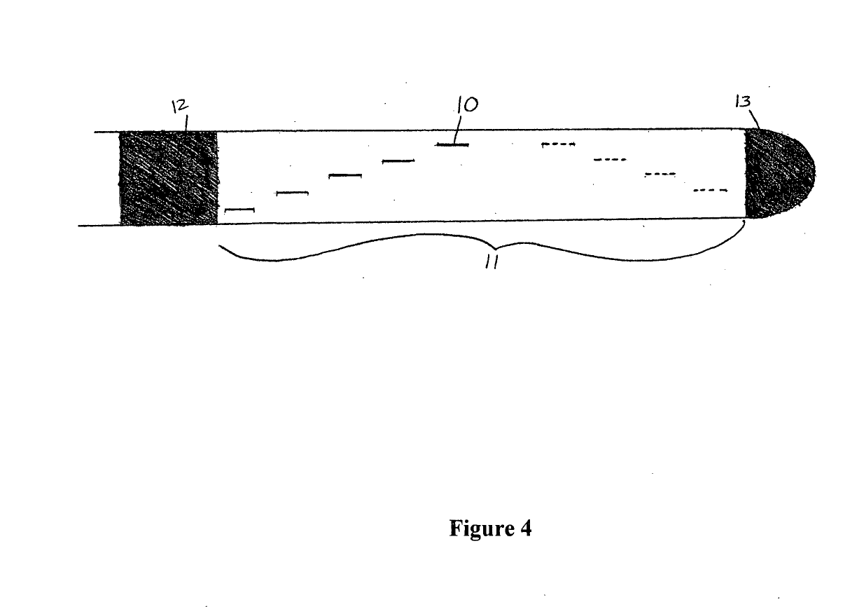

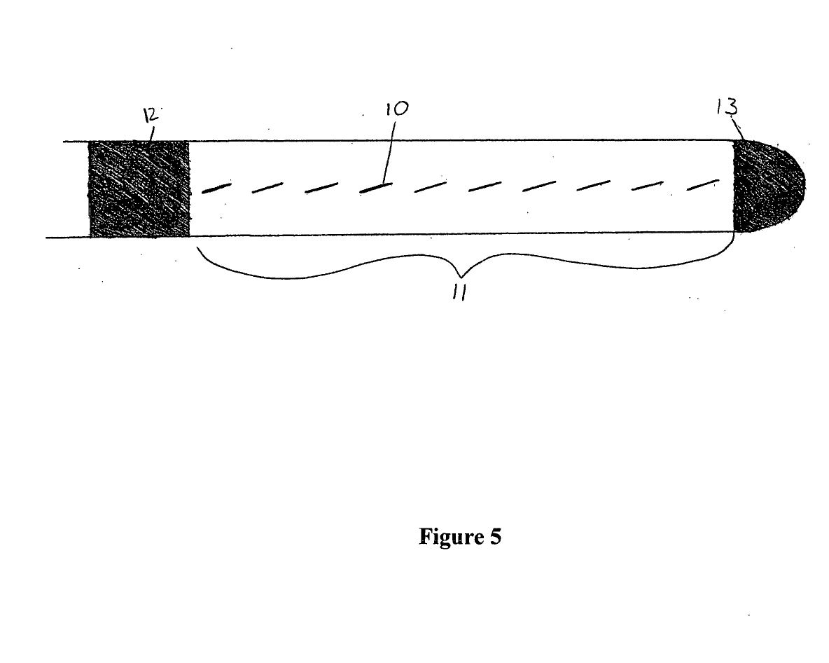

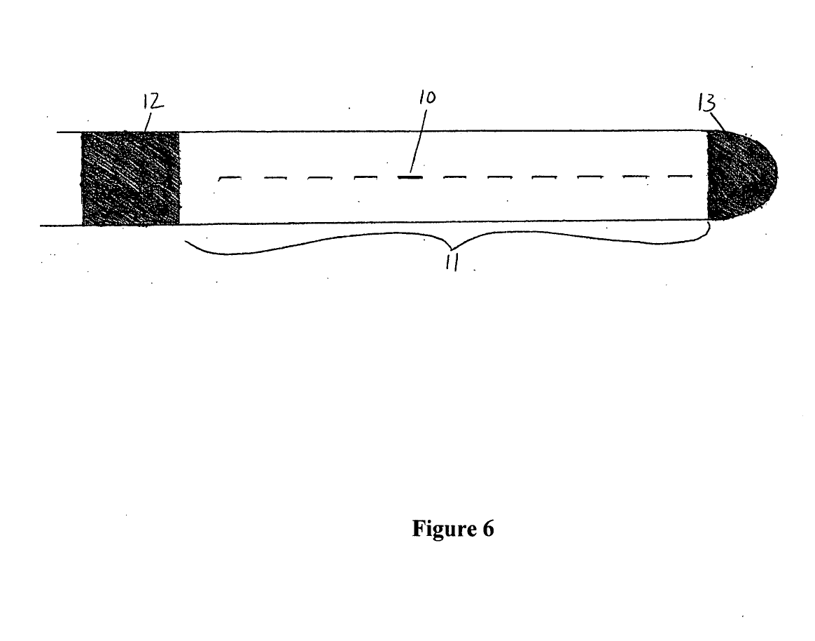

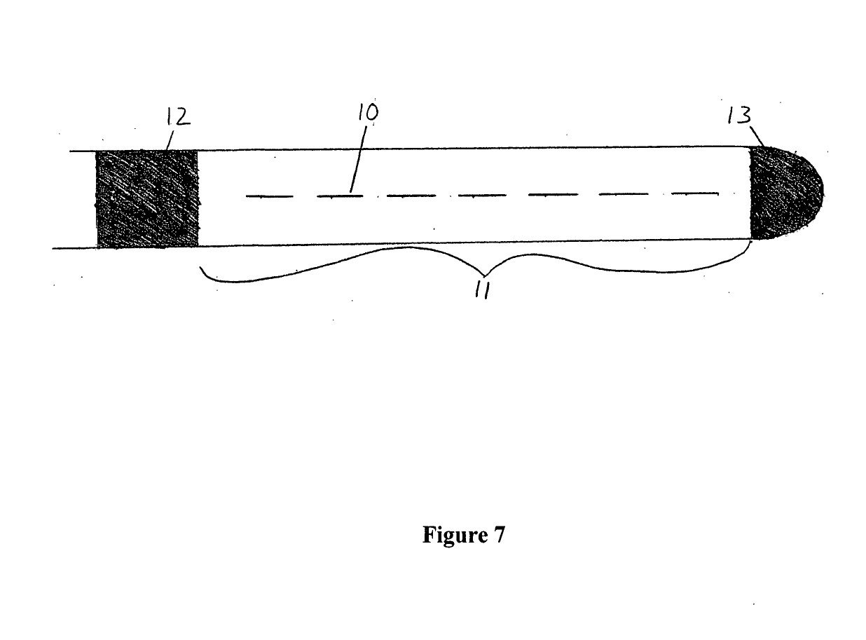

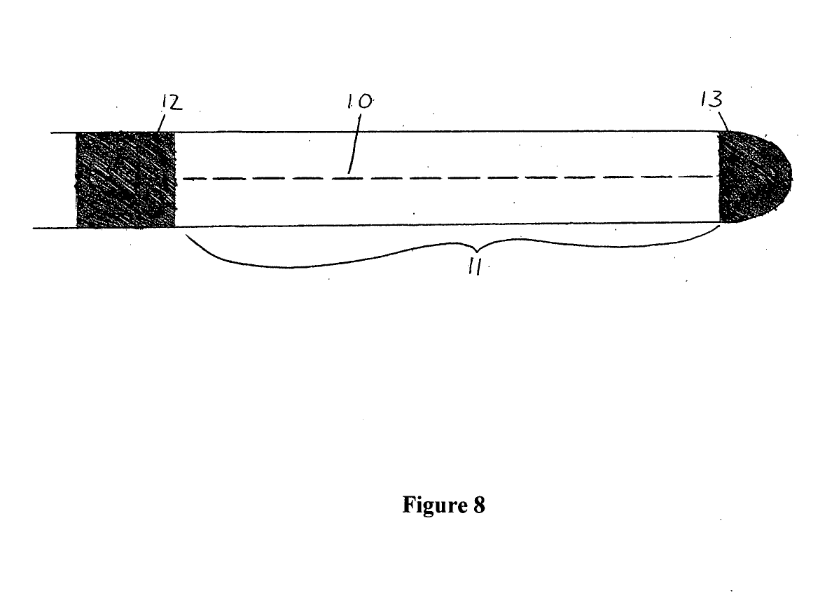

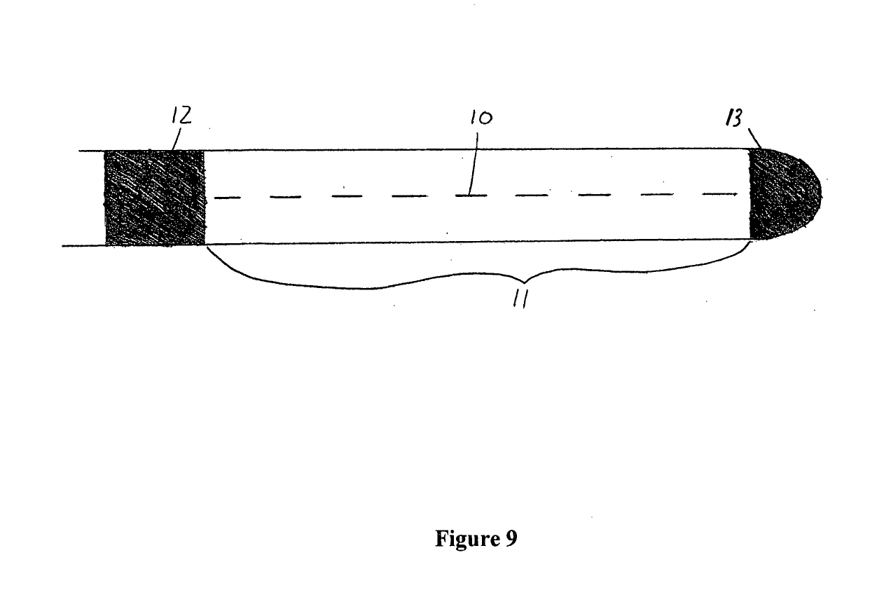

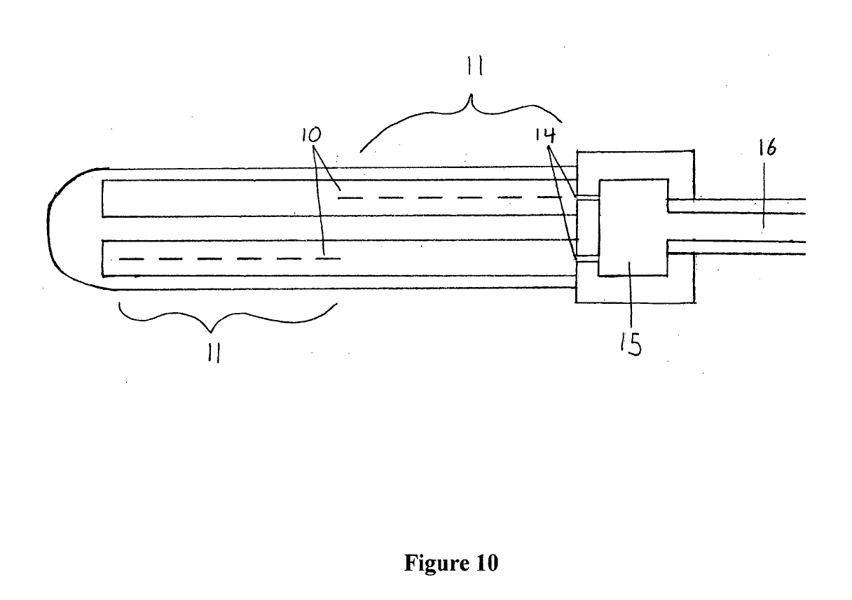

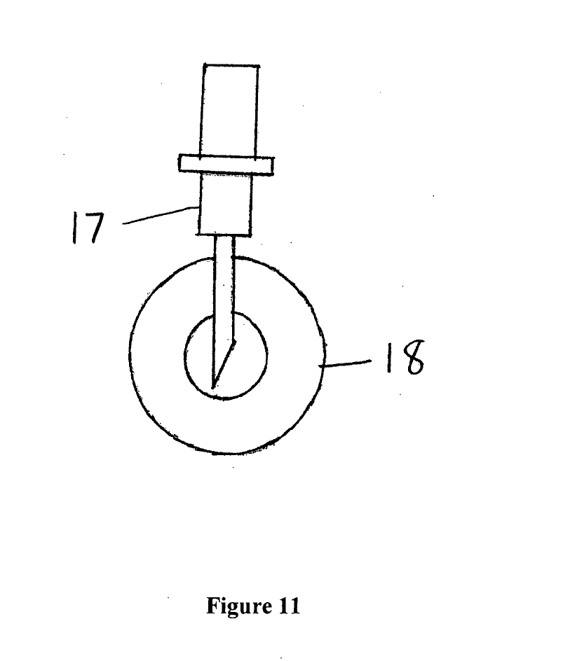

The figures exemplify certain embodiments of the invention. FIG. 1 is an example of a catheter of the current invention (1) with an infusion segment (2) and connected to an elastomeric infusion pump (7) with a liquid fill port (8) through an infusion pump connector (3), a flow restrictive element (4), a flow restrictor connector (5), and a filter (6). FIG. 2 shows examples of cross sections of catheters of the invention. Exemplified are a single-lumen catheter (A and B) and a dual-lumen catheter (C). FIGS. 3-9 show different embodiments of a catheter of the invention with delivery slits (10) of different sizes and arrangements in an infusion segment (11) and with a printed band at the proximal end of the infusion segment (12) and a closed distal end (13). FIG. 10 shows an example of a dual lumen catheter according to certain embodiments, also illustrating delivery slits (10) in two infusion segments (11) for fluid delivery from each of the two lumens. Also exemplified in FIG. 10 are flow restrictive channels (14), a high pressure fluid manifold (15), and an infusion set tubing (16). FIG. 11 exemplifies a method for making a delivery slit in a catheter of the invention by piercing with a lancet (17) through the catheter wall (18).

### 5.1 Catheters of the Invention.

Catheters of the invention, in certain embodiments, comprise a structure in the general shape of a tube. A catheter of the invention, in certain embodiments, comprises a wall and an inner lumen through which fluid may flow into the catheter for delivery to a patient. In certain other embodiments, a catheter of the invention comprises a closed end. In certain other embodiments, a catheter of the invention comprises more than one lumen (multi-lumen catheter), for example, two inner lumens through which a fluid may flow for delivery to a patient, or three, or four, or more inner lumens. An inner lumen of a catheter of the invention, in certain embodiments, may have a cross-section in a shape that facilitates flow and delivery of fluids to a patient, most preferably round. In certain other embodiments, an inner lumen of a catheter of the invention may have a cross-section that is round, oval, square, irregular, triangular, egg-shaped, and any combination of these shapes or a combination where one or more sections of a catheter have a cross-section of one shape and one or more other sections have a cross-section with another shape.

A catheter of the invention, in certain embodiments, comprises an opening for supplying a fluid into the catheter (“supply port”). In certain embodiments, a supply port of a catheter of the invention may be connected to or fused with another device (for example, an infusion pump) which supplies fluid into the catheter. In certain other embodiments, there may be two, three, four or more supply ports, wherein each supply port is capable of supplying fluid into one, two or more lumens in a multi-lumen catheter. In certain embodiments, a catheter of the invention comprises slits through which a fluid is delivered from the catheter to a patient (“delivery slits”). A catheter of the invention, in certain embodiments, can be used subcutaneously, within an orthopedic joint, at a nerve juncture, or within a body cavity or vessel. In certain preferred embodiments, the delivery slits of a catheter of the invention comprise micro slit valves through which fluid is delivered to a patient. In certain embodiments, delivery slits of a catheter of the invention may be arranged in clusters in a segment of the catheter (infusion segment) and delivery slits may be spaced regular or irregular along the longitudinal axis and/or around the perimeter of the catheter. In certain embodiments, there are two or more infusion segments for an individual catheter, with each infusion segment being in fluid communication with one or two dedicated catheter lumens.

A delivery slit in a catheter of the invention, in certain embodiments, can function as a pressure responsive valve (valve function) and/or as a flow restrictive element (restrictor function). A delivery slit of a catheter of the invention, in certain embodiments, can fulfill a valve function and a restrictor function at a lower cost as compared to a catheter design in which both functions must be fulfilled by distinct design features, especially where such distinct features require distinct components.

Catheters of the current invention, in certain embodiments, are simple to use. In certain preferred embodiments, catheters of the invention are also cost effective to produce and preferably of unitary construction, for example, by using a small number of components, for example, one, two, three, four or five components. A catheter of the invention, in certain preferred embodiments, has a simple design and a good reliability. In certain other preferred embodiments, a catheter of the invention is designed so that it may be produced at low cost. A catheter of the invention, in certain embodiments, facilitates uniform or substantially uniform liquid flow at low flow rates, for example, uniform or substantially uniform liquid flow through some or all of the delivery slits of the catheter. A catheter of the current invention according to certain embodiments facilitates some or all of these features due to its design, for example, due to its diameter, due to the length, due to the length of the infusion segment, and due to the number, shape and size of delivery slits in the catheter wall in the infusion segment.

In certain preferred embodiments, a catheter of the invention comprises a plurality of delivery slits to facilitate a more uniform fluid delivery through the slits throughout the infusion segment of a catheter. In certain embodiments, a slit in a catheter of the invention is preferably short or small. In certain preferred embodiments, a slit in a catheter of the invention does not reach through the catheter wall in an orientation that is perpendicular to the longitudinal axis of the tube, but rather at an angle of less than ninety degrees relative to the longitudinal axis of the tube in any orientation, for example, at 0 to 90 degrees, or about 0 to about 90 degrees, at 0 to 60 degrees, or about 0 to about 60 degrees, at 0 to 45 degrees, or about 0 to about 45 degrees, at 0 to 30 degrees, or about 0 to about 30 degrees, or 20 to 60 degrees, or about 20 to about 60 degrees.

In other words, the slits are generally linear relative to the longitudinal axis of the tube under these embodiments and preferably at an angle of less than 90 degrees relative to the longitudinal axis, with the most preferred angle being zero degrees or about zero degrees.

In certain other embodiments, a catheter of the invention does not comprise certain elements or aspects. For example, according to certain embodiments, a catheter of the invention does not comprise one, two, three, or more of a spring, a coil spring, a spring in the lumen of the catheter, a coil spring in the lumen of the catheter, a spring in the catheter wall, a coil spring in the catheter wall, a spring that extends through the entire catheter, a spring that extends through parts of the catheter, a spring that extends through an infusion segment of the catheter, a spring that reaches the tip of the catheter, a guide wire, a membrane, a tubular membrane, a flat membrane, a spiral membrane, a hollow membrane, a flow distribution element, an inserted flow distribution element, a fiber, a cloth, a wick, a sponge, and/or a hollow fiber, and any combination of these elements.

### 5.2 Flow Rates of Catheters of the Invention.

A catheter of the invention, in certain embodiments, can be used for a flow rate from about 0.5 milliliters per hour (ml/hr) to about 10 ml/hr, or from 0.5 ml/hr to 10 ml/hr, or from 1 ml/hr to 5 ml/hr, or from about 1 ml/hr to about 5 ml/hr, and more preferably from 1 ml/hr to 3 ml/hr, or from about 1 ml/hr to about 3 ml/hr. For example, a catheter of the invention, in certain embodiments, may be used with an infusion pump with a flow rate from about 0.5 ml/hr to about 10 ml/hr, or from 0.5 ml/hr to 10 ml/hr, or from 1 ml/hr to 5 ml/hr, or from about 1 ml/hr to about 5 ml/hr, and more preferably from 1 ml/hr to 3 ml/hr, or from about 1 ml/hr to about 3 ml/hr. In certain other embodiments, a catheter of the invention can be used for a flow rate from 10 ml/hr to 400 ml/hr, or about 10 ml/hr to about 400 ml/hr, or from 10 ml/hr to 200 ml/hr, or about 10 ml/hr to about 200 ml/hr, or from 10 ml/hr to 100 ml/hr, or about 10 ml/hr to about 100 ml/hr. The flow rate of a catheter can be determined, for example, by measuring the total amount of fluid that flows out of a catheter through all openings and passages over a period of time, for example, ml per hour.

In certain embodiments, a catheter of the invention can be used with an infusion device, for example an elastomeric infusion pump. When used with an infusion device, a catheter of the invention, in certain embodiments, will mostly flow at slightly less than what the infusion device would flow at without the catheter. In certain embodiments, a catheter of the invention adds a backpressure to the fluid circuit, which can somewhat reduce the flow rate. In general, the flow rate of an infusion device may be reduced by about 2-10 percent when compared to an infusion device without a catheter attached. Also in general, when used in an artery, the flow rate may be subject to some arterial backpressure that could slow down the flow rate, while the venous system and typical wound sites are less likely to slow the flow rate measurably.

Flow rates useful for chemotherapy applications may be from about 0.1 ml/hr to about 10 ml/hr, and for post operative pain management applications, flow rates may be from about 1 ml/hr to about 5 ml/hr. Flow rates for intravenous administration of antibiotics commonly range from about 10 ml/hr to 200 ml/hr and are generally around 100 ml/hr, and may be up to 400 ml/hr or even higher.

When using a catheter of the invention, for example, with an elastomeric infusion pump, a flow rate is generally achieved by balancing the pressure within the infusion bladder with the backpressure produced by all flow restrictive elements of the fluid circuit. For example, a narrow capillary or tubing segment may be used to restrict flow. A catheter backpressure is a part of the overall backpressure, although in general not a significant part. A catheter with design features that restrict flow, for example a tubular cylinder membrane, require a higher pressure for delivery of a fluid and an infusion device used with such a catheter requires higher pressure which may increase the risk of leakage and material failure and fatigue. Also, it is generally more expensive to make a higher pressure generating infusion pump because thicker or higher modulus elastomeric infusion bladders are needed. This also makes it more difficult for practitioners to fill the pumps with liquid prior to the start of infusion. Examples of design features of a catheter that can restrict flow of the catheter are features that obstruct the catheter lumen, or make the lumen smaller, or impede the flow of fluid in the catheter lumen, for example, a spring, a coil spring, a spring in the lumen of the catheter, a coil spring in the lumen of the catheter, a spring in the catheter wall, a coil spring in the catheter wall, a spring that extends through the entire catheter, a spring that extends through parts of the catheter, a spring that extends through an infusion segment of the catheter, a spring that reaches the tip of the catheter, a guide wire, a membrane, a tubular membrane, a flat membrane, a spiral membrane, a hollow membrane, a flow distribution element, an inserted flow distribution element, a fiber, a cloth, a wick, a sponge, and/or a hollow fiber, and any combination of these elements.

### 5.3 Cracking Pressures of Catheters of the Invention.

A catheter of the invention, in certain embodiments, has a cracking pressure that is sufficiently high to prevent leakage after priming of the catheter. In certain embodiments, the cracking pressure of a catheter of the invention is about 0.1 psi to about 15 psi, or 0.1 psi to 15 psi, or about 0.1 psi to about 6 psi, or 0.1 psi to 6 psi. In certain preferred embodiments, the cracking pressure of a catheter of the invention is from 0.2 psi to 2 psi, or from about 0.2 psi to about 2 psi, more preferably from 0.3 psi to 1 psi, or from about 0.3 psi to about 1 psi. In certain other embodiments, the cracking pressure of a catheter of the invention is at least 0.1 psi, or at least 0.2 psi, or at least 0.5 psi, or at least 1.0 psi.

In certain embodiments, a catheter of the invention is capable of achieving a balanced distribution of flow through delivery slits of the catheter regardless of the position the catheter is in, for example, if the catheter is in a horizontal position, a vertical position, if the catheter is straight, or if the catheter is bent. In certain embodiments, the cracking pressure of a catheter is at least equal to, or about equal to, the associated head pressure of water, for example, 0.432 psi per foot of head. In certain embodiments, the cracking pressure of a catheter is at least 10 percent higher than the associated head pressure of water, or at least 20 percent, or at least 30 percent higher. In certain embodiments, the cracking pressure of a catheter is at least equal to, or about equal to, the head pressure of the fluid in the length of the catheter. In certain embodiments, the cracking pressure of a catheter is at least 10 percent higher than the head pressure of the fluid in the length of the catheter, or at least 20 percent, or at least 30 percent higher.

### 5.4 Infusion Segments of Catheters of the Invention.

A catheter of the invention, in certain embodiments, is able to distribute fluid in a uniform or substantially uniform manner over an entire infusion segment of the catheter or over more than 70 percent of an infusion segment of the catheter. An infusion segment of a catheter of the invention comprises a segment of the catheter from a first delivery slit to a last delivery slit, typically with multiple delivery slits in between. In certain embodiments, a catheter of the invention comprises one infusion segment; in certain other embodiments, it comprises two, three, four, five, or more infusion segments. The distribution of delivery slits in an infusion segment of a catheter of the invention, in certain embodiments, is even or uneven, for example, delivery slits may be spaced evenly or unevenly, delivery slits may be along one line or along a plurality of lines, delivery slits may be on one side of a catheter of the invention or on more than one side.

An infusion segment of a catheter of the invention, in certain embodiments, has a length from about 0.5 inches to about 20 inches, or from 0.5 inches to 20 inches, more preferably from about 1 inch to about 15 inches, or 1 inch to 15 inches, and most preferable from about 2.5 inches to about 10 inches, or from 2.5 inches to 10 inches.

In certain other embodiments, an infusion segment of a catheter of the invention is at a distal tip (in other words distal from the end where fluid is supplied into the catheter for delivery to the patient) of the catheter. In certain other embodiments, an infusion segment is at a distance from a distal tip, for example, from about 1 to about 10 inches, or from 1 to 10 inches, or from about 2 to about 6 inches, or from 2 to 6 inches.

### 5.5 Diameter of Catheters of the Invention.

A catheter of the invention, in certain embodiments, has an outside diameter and an inside diameter. An outside diameter of a catheter of the invention, in certain embodiments, is about 0.020 to about 0.125 inches, or 0.020 to 0.125 inches, or about 0.030 to about 0.100 inches, or 0.030 to 0.100 inches, or about 0.040 to about 0.080 inches, or 0.040 to 0.080 inches, or about 0.030 to about 0.065 inches, or 0.030 to 0.065 inches. An inside diameter of a lumen of a catheter of the invention, in certain embodiments, is about 0.001 to about 0.099 inches, or 0.001 to 0.099 inches. In certain preferred embodiments, a catheter of the invention has an outside diameter of 0.042 inches, or about 0.042 inches, and an inside diameter of 0.018 inches, or about 0.018 inches.

In certain embodiments, the outside diameter of a catheter of the invention can be measured anywhere along the catheter, or anywhere in an infusion segment of the catheter. In certain other embodiments, the outside diameter of a catheter of the invention may vary along the catheter, for example, by at least ±5 percent, or by at least ±10 percent, or by at least ±25 percent, or by at least ±50 percent, or by ±0 to 50 percent, or by ±0 to 25 percent, or by ±5 to 25 percent.

In certain preferred embodiments, a catheter of the invention has an outside diameter that is sufficiently small so that fluid exiting the catheter may access all or substantially all areas around the circumference of the catheter. In these preferred embodiments, a catheter of the invention obviates the need to radially space the delivery slits, for example, it is not necessary under these embodiments to space delivery slits apart by certain degrees around the circumference of the catheter (for example, by 120 degrees).

### 5.6 Delivery Slits in Catheters of the Invention.

### 5.6.1 Location of Delivery Slits.

In certain embodiments, a delivery slit of a catheter of the invention prevents or minimizes the flow of fluids into the catheter lumen. A delivery slit is positioned, in certain embodiments, to prevent or minimize the flow of fluids into the catheter lumen. A delivery slit is positioned, in certain preferred embodiments, to prevent or minimize the flow of fluids into the catheter lumen by placing it in a convex portion of a catheter. In certain less preferred embodiments, a delivery slit is placed in a flat portion, a concave portion, a thinned portion, and/or a thickened portion of the catheter. In certain other embodiments, a delivery slit of a catheter of the invention is oriented along the axis of the catheter along which fluid flows in the catheter. In certain other embodiments, delivery slits of a catheter of the invention are spaced radially along the catheter, in certain other embodiments, they are not spaced radially.

### 5.6.2 Size of Delivery Slits.

In certain embodiments, a delivery slit of a catheter of the invention is longer when measured on the outside of the catheter and shorter when measured on the inside. A delivery slit according to these embodiments facilitates an improved distribution of fluid flow on the outside of the catheter, for example, along an infusion segment of a catheter of the invention, while not increasing the overall liquid flow rate of the catheter.

In most preferred embodiments, a delivery slit of a catheter of the invention is of a short length, for example, to achieve a desired cracking pressure. The length of a slit is inversely proportional to the cracking pressure. A long slit is associated with a low cracking pressure, a medium length slit is associated with medium cracking pressure, and a short slit is associated with a high cracking pressure. The depth of a slit also affects the cracking pressure of a slit. For example, if the wall thickness of the catheter tubing constitutes the depth of a slit, a thin wall tubing produces low cracking pressures, medium wall thickness tubing produces medium cracking pressures, and high wall thickness tubing produces high cracking pressures.

In certain embodiments, the length of a delivery slit of a catheter of the invention is from about 0.002 to about 0.060 inches, or from 0.002 to 0.060 inches, from about 0.003 to about 0.040 inches, or from 0.003 to 0.040 inches, from about 0.004 to about 0.030 inches, or from 0.004 to 0.030 inches, from about 0.005 to about 0.020 inches, or from 0.005 to 0.020 inches, from about 0.006 to about 0.016 inches, or from 0.006 to 0.016 inches, from about 0.006 to about 0.010 inches, or from 0.006 to 0.010 inches, and most preferred about 0.008 inches or 0.008 inches.

### 5.6.3 Spacing of Delivery Slits.

In certain embodiments, the spacing of delivery slits of a catheter of the invention is from about 0.05 to about 1 inch apart, or from 0.05 to 1 inch, from about 0.1 to about 0.5 inches, or from 0.1 to 0.5 inches, from about 0.1 to about 0.2 inches, or from 0.1 to 0.2 inches, and most preferably about 0.125 inches or 0.125 inches apart. In certain embodiments, an infusion segment of a catheter of the invention has one slit per 0.1 inches, or at least one slit per 0.1 inches, or one slit per 0.2 inches, or at least one slit per 0.2 inches, or one slit per 0.5 inches, or at least one slit per 0.5 inches, or one slit per 1 inch, or at least one slit per 1 inch, or one slit per 2 inches, or at least one slit per 2 inches. In certain embodiments, the spacing of delivery slits in an infusion segment of a catheter of the invention may be regular or irregular, or in a pattern where two, three, four or more of the above spacing of slits are found in an infusion segment.

In certain other embodiments, delivery slits are spaced by degrees around the radius of the catheter wall, for example, two consecutive slits may be spaced by 5 degrees, or at least 5 degrees, or by 10 degrees, or at least 10 degrees, or by 15 degrees, or at least 15 degrees, or by 20 degrees, or at least 20 degrees. In certain embodiments, delivery slits in an infusion segment may be on one, two, three or four sides of the catheter, for example, spaced by 90 degrees or 180 degrees around the radius of the catheter wall. For example, delivery slits may be on one, two, three or four sides of a catheter so that a group of slits is on each of one, two, three or four sides and wherein a group of slits is at least 2 slits, or at least 4, or at least 8, or at least 12, or from 2 to 20, or from 2 to 10.

### 5.6.4 Number of Delivery Slits.

In certain preferred embodiments, a catheter of the invention has from about 3 to about 300 delivery slits, or from 3 to 300, or from about 5 to about 200, or from 5 to 200, or from about 8 to about 120, or from 8 to 120, or from about 15 to about 80, or from 15 to 80, or from about 25 to about 50, or from 25 to 50. In certain embodiments, a catheter of the invention has from about 5 to about 50 delivery slits, or from 5 to 50, or from about 10 to about 50, or from 10 to 50, or from about 15 to about 50, or from 15 to 50, or from about 30 to about 80, or from 30 to 80, or from about 50 to about 100, or from 50 to 100. In certain embodiments, a catheter of the invention has at least 5 delivery slits, or at least 10, or at least 15, or at least 20, or at least 30, or at least 50, or at least 100. Each and every of the above numbers of delivery slits may be found in an infusion segment of a catheter of the invention, or, in certain other embodiments, in a catheter in its entirety.

### 5.6.5 Shape and Orientation of Delivery Slits.

In certain embodiments, a delivery slit of a catheter of the invention may be linear, curved, angled, a semi circle, cross shaped, or any other shape. In certain preferred embodiments, a delivery slit is linear. In certain preferred embodiments, a delivery slit of a catheter of the invention has an angular shape (angular delivery slit), in other words, the portion of the slit on the outside of the catheter is longer than the portion of the slit in the lumen of the catheter. A delivery slit with an angular shape can be formed, in certain embodiments, with an angles cutting or slitting instrument. An angular delivery slit, in certain embodiments, facilitates greater linear fluid coverage on the outside of the catheter. In certain preferred embodiments, it is desirable to have as much wetted surface as practical on the outside of a catheter for purposes of regional anesthesia (RA), for example, the delivery of local anesthetic along the length of a wound site during surgery is aided through a wetted catheter surface.

### 5.7 Methods of Using a Catheter of the Invention.

A catheter of the invention, in certain embodiments, can be used to deliver a medical fluid to a specific portion of the body, for example, into a blood vessel, an internal organ, the gastrointestinal tract, the brain, a kidney, the liver, the area containing the cerebral spinal fluid, a subcutaneous region, or any other portion of the body. A catheter of the invention, in certain embodiments, can be used to drain or sample bodily fluids from the body, such as urine from the bladder, cerebral spinal fluid from the brain, or gastrointestinal track contents.

A catheter of the invention, in certain preferred embodiments, can be used in the area of regional anesthesia (RA). Regional anesthesia is used to compliment or replace traditional general anesthesia. An example of regional anesthesia is that of post surgical pain relief systems. Such systems are typically used to alleviate pain proximal to the area upon which surgery was performed. Often, this is at the point of a surgical incision site. In other instances, it may be in the general vicinity of the incision site. The steady flow of an RA medication over a period of time reduces the need for systemically acting pain relieving drugs. The RA medication can optionally include an antibiotic medication to prevent infection to the wound site. RA pain relief systems generally involve the use of a pump and a catheter to deliver fluid to the patient. The pump can deliver a medicinal fluid to the patient via a catheter according to the present invention. Suitable pumps include those suitable for delivering fluid under pressure. Typical pumps include electromechanical types, powered by small electrical motors, elastomeric infusers, pressurized gas infusers, vacuum powered pumps, and the like. Optionally, a pump can be omitted, and a container of medical fluid can be placed at an elevation higher than that of the medication delivery site. In this case, the head pressure of the fluid provides the pressure for the delivery of the medical fluid.

A catheter of the invention, in certain embodiments, can be used to deliver a medication to a wound site. If the medical fluid to be delivered is needed at only one specific point in the body, then a catheter according to the invention with from one to about three delivery slits at the distal end may be all that is required. If medical fluid is to be delivered over an extended length of the distal (or other portion) of the catheter of the invention, then a more extended distribution of delivery slits is desired. If uniform or substantially uniform delivery of the medical fluid at low flow rate and low pressure along the length of the infusion segment of the catheter of the invention is important, then a catheter of the invention with those features should be used.

In certain embodiments, a catheter of the invention is used with an infusion pump and a flow restrictor combination to achieve low flow rates as desired. The catheter lumen pressure will build up until the cracking pressure is reached, at which time fluid flow will begin. If very low delivery pressure is desired, then low cracking pressure slits are needed.

### 5.8 Methods of Making a Catheter of the Invention.

A catheter of the current invention, in certain embodiments, is made with a method of the invention for making a catheter. A method of the invention for making a catheter, in certain embodiments, comprises using a lancet to generate a delivery slit in a catheter tubing. In certain embodiments, a method for making a catheter of the invention comprises securing catheter tubing; and/or piercing the wall of catheter tubing with one or more of a lancet, a needle, a pointed needle, a round needle, a blade, and/or a beveled blade; and/or generating a delivery slit; and/or repeating the piercing step to generate a desired number of delivery slits in said catheter tubing, for example, repeating it 5 times or at least 5 times, 10 times or at least 10 times, 20 times or at least 20 times.

The present invention is further illustrated by the following examples, which are not intended to be limiting in any way whatsoever.

### EXAMPLES

### Example 1

### Making a Catheter of the Invention

This specification describes the method by which the Catheter Tubing, P/N 40029-X, is processed to become the Zone Catheter, P/N 59014-X.

1.1 Equipment and Supplies.

The following equipment and supplies are used to make a catheter: (a) BD Ultra Fine 33 Gauge Lancets and Lancet Caps, BD product #322057; (b) a Lancing Fixture with Indexing template including #12-24 machined stainless steel screws and machined lancet caps; (c) a Sprint LC Multi Air Tester, Industrial Data Systems Model LC-PF, fitted with a touhy borst or other connection fitting for catheters, connected to regulated compressed air (compressed air supply should be filtered both at the main supply tank and at point-of-use with a minimum of a particulate filter and coalescing filter); (d) lancet binning fixture setup with continuity tester; (e) 0.2 micron point-of-use filter; (f) natural rubber-free gloves or finger cots; (g) Catheter Tubing, P/N 40029-X.

1.2 Set-Up and General Requirements.

Steps are carried out inside a Class 100,000 or Class 8 Clean Room or better, and while wearing natural rubber-free gloves or finger cots during the assembly process. The area should be kept at 68° F. to 77° F. or close thereto. A Sprint LC Multi Air Tester is set up per the instructions in the Owner's Guide. The tester is connected to regulated and filtered compressed air. Press NEXT to highlight Test Parameters if not already highlighted, then press ENTER. At the Test Parameters screen of the Tester, verify program parameters are set as follows: Test Type—FLOW; Test Pressure—2.500 psi; Pressure Error—0.0 psi; Coupling—0.0 s; Fill Time—2.0 s; Test Time—500 s; Dump—NO.

The Lancing Fixture and Lancets are set up as follows. Insert machined lancet cap into recess on lancing fixture. The lancet cap must be drilled and tapped through the bottom (closed end) and have a drilled through hole that has been drilled with a 0.032″ diameter bit and reamed with a 0.035″ diameter reamer perpendicular to its length and approximately 0.424″ from the top (open end). Place a few wraps of PTFE tape around a stainless steel screw that has been precision machined with a flat at the end of the threads and thread it through the through hole beneath the recess and into the lancet cap. Care should be taken to ensure that no PTFE tape extends up past the top flat portion of the screw. Align the lancet cap so that the through hole is parallel to the length of the lancing fixture. Close the lancet cover of the lancing fixture, capturing the lancet cap in place and securing it. Secure cover with clamps or screws as appropriate. Secure lancing fixture to table with clamps or screws as appropriate and secure indexing template to top of fixture, such that it runs parallel along the top of the fixture and is in direct contact with the lancing cap.

The Bin Lancets are arranged by length as follows. The length of each individual lancet must be determined before it can be used so the insertion depth into the Catheter Tubing can be precisely controlled. Place lancet into first hole position of lancet binning fixture which corresponds to a lancet length of 0.436″. Insert continuity tester probe into back (open) end of the lancet, allowing it to contact the metal wire that is present inside of the lancet. If tester lights up, showing a continuous circuit, discard lancet. This lancet is longer than 0.436″ and cannot be used. If tester does not light up, move lancet to next hole position which corresponds to a lancet length of 0.435″. If the tester lights up, the lancet is placed in 0.435″ bin. If the tester does not light up, move the lancet to the next hole position. Continue to test lancet in each hole (must proceed in descending order: 0.436″, 0.435″, 0.434″, etc.) until the lancet is found to be in a hole which causes the tester to light up. Place the lancet into the bin that corresponds to the hole position in which the tester lit up. If all hole positions have been tried and the tester has not lit up, discard the lancet. The lancet is shorter than 0.428″ and cannot be used.

Before lancing of Catheter Tubing can begin, a correct pairing of lancet and lancet cap must be made. Choose a lancet cap. Insert a stainless steel rod that is 0.031″ OD into through hole. Engage the screw so that is just lightly applies pressure to the stainless steel rod, but does not bend it. Remove stainless steel rod and measure the distance from the top edge of the cap to the surface of the screw using a depth micrometer. Subtract 0.008″ from this depth measurement. The result is the length of the lancet that must be used. (For example: after inserting a stainless steel rod and engaging the screw into a lancet cap, the depth to screw surface is measured to be 0.436″. 0.436″−0.008″=0.428″ Lancets from the 0.428″ bin should be used.) If after setup and measurement has been performed, the needed lancet length is not one of the available bins, the lancet cap is out of range and must be discarded.

1.3 Procedure.

Verify that the Sprint Tester is on and has been properly setup with 0.2 micron point-of-use filter in line with the air flow as it exits the tester and before it enters the catheter tubing. Take catheter to be processed and insert proximal (open) end into connection fitting (touhy borst or equivalent) on the Sprint LC Multi Air Tester (or equivalent). Secure catheter tubing and press the start button on the tester. Verify that flow reading is less than or equal to 0.05 sccm. If a flow reading is found, verify that no leaks exist in the connection fittings. If no leaks are found, the catheter tubing is defective and has an unanticipated hole. Discard catheter tubing. Insert Distal (closed) end of catheter tubing into machined hole in a lancet cap that is secured in the lancing fixture. Advance the catheter tubing so that it extends through the first open hole and just into the second, without protruding out past the outer wall of the lancet cap.

Insert the lancet into the lancet cap fully, allowing the metal portion to pierce the catheter tubing. The lancet must be oriented such that the bevel is parallel to the catheter tubing and the lancet makes a slit in the tubing that is parallel to its length. The insertion of the lancet must be a very deliberate straight-on stroke. The lancet cap will help orient the lancet, but it is critical that the operator start the stroke straight, so as not to hit the wall of the lancet cap and so that the lancet pierces the catheter tubing correctly. The lancet must also be pushed firmly and fully into the lancet cap. Care should be taken to ensure that the lancet is fully bottomed out in the lancet cap. A new lancet should be used at the start of each shift, but may be changed more often as necessary to properly pierce the catheter tubing. Remove the lancet from the lancet cap while simultaneously watching the flow reading on the tester. Verify that the flow number increases. The number will tend to increase by a large amount during the lancing of the initial holes, and taper off to a much lower number by the time the last few holes are lanced. If the flow number does not increase, inspect lancet for damage and verify that the catheter tubing is aligned straight through the lancet cap. Re-adjust tubing or replace lancet if necessary. Again insert the lancet into the lancet cap to pierce the catheter tubing. If the flow number still does not increase, discard tubing and lancet. Advance the catheter tubing by one position (⅛″) as read on the indexing template, keeping the tubing as straight as possible on the template to ensure accurately spaced holes. Repeat the steps in this paragraph to continue to insert holes in the catheter tubing at ⅛″ spacing.

Stop indexing catheter tubing and inserting holes when the tip of the catheter tubing has reached the desired stop marking on the indexing template corresponding to the correct Zone length. A final hole should be inserted while the catheter tubing is in its final position with the tip of the catheter on the final indexing position. After the final hole has been lanced, the air flow number should be read to verify that the number falls between 70 and 117 sccm. If air flow is out of range, tubing should be discarded and the setup should be checked for problems. One potential problem could be that the set pressure on the Sprint tester has drifted. Verify that pressure is set to 2.500±0.05 PSI. If no problems are detected, lancing may proceed.

1.4 Quality Control Inspection.

To be performed after all parts in a batch have been manufactured. Samples which pass inspection may be replaced into the lot. Samples which fail inspection must be discarded or reworked. Pull samples for a visual inspection. Examine sample for particulate visible to the unaided eye in or on Catheter, for manufacturing defects, for obvious discoloration, and for catheters kinked or damaged. Conduct a dimensional inspection. Catheter specifications (table 1):

| | | | Spacing From | |
|---|---|---|---|---|
| Part | Length | Outside Diameter | Tip to Band | |
| Number | (+/− 0.25″) | (+/− 0.002″) | (+/− 0.025″) | Slit Spacing |
| 59014-2 | 24.00″ | 0.031″ | 2.50″ | 0.125 inches |
| 59014-3 | 24.00″ | 0.031″ | 5.00″ | 0.125 inches |
| 59014-4 | 24.00″ | 0.031″ | 10.00″ | 0.125 inches |

Pull samples randomly for inspection of dimensions. Measure the dimensions of the catheter. The following would be considered defects: the length is not 24.00″±0.25″; the catheter does not have at least one hole every 0.5″; any hole is further than 0.125″ past end of black band that signifies the infusion length; infusion segment length not within specification.

The present invention is not to be limited in scope by the specific embodiments described herein, which are intended as single illustrations of individual aspects of the invention, and functionally equivalent methods and components are within the scope of the invention. Indeed, various modifications of the invention, in addition to those shown and described herein, will become apparent to those skilled in the art from the foregoing description. Such modifications are intended to fall within the scope of the appended claims. All cited publications, patents, and patent applications are herein incorporated by reference in their entirety for any purpose.

What is claimed is:

1. A catheter for delivering a fluid to a patient comprising a tube, said tube comprising a lumen and wall with delivery slits in said wall, wherein said tube has an outside diameter of about 0.020 to about 0.125 inches, wherein said delivery slits are in an infusion segment of the tube, wherein said infusion segment includes at least one slit per inch, wherein said catheter has a cracking pressure of at least 0.1 psi, wherein said catheter does not comprise a spring, a membrane or a wick in said lumen.

2. The catheter according to claim 1, wherein said cracking pressure comprises at least 0.2 psi.

3. The catheter according to claim 1, wherein said infusion segment comprises at least 10 slits.

4. The catheter according to claim 1, wherein said infusion segment comprises two infusion segments.

5. The catheter according to claim 4, wherein said infusion segments are on different sides of the catheter.

6. The catheter according to claim 1, wherein said catheter has at least two lumens and wherein each of said two lumens has an infusion segment with delivery slits, and wherein said catheter does not comprise a spring, a membrane or a wick in said two lumens.

7. The catheter according to claim 6, wherein a flow restrictor is associated with each of said two lumens.

8. The catheter according to claim 6, wherein said catheter has a cracking pressure of at least 0.2 psi.

9. The catheter according to claim 6, wherein said cracking pressure comprises at least 0.2 psi.

10. The catheter according to claim 6, wherein said infusion segment comprises at least 10 slits.

11. The catheter according to claim 6, wherein said infusion segment comprises two infusion segments.

12. The catheter according to claim 11, wherein said infusion segments are on different sides of the catheter.

13. A method for delivering a fluid to a patient comprising delivering said fluid with a catheter, said catheter comprising a tube, said tube comprising a lumen and wall with delivery slits in said wall, wherein said tube has an outside diameter of about 0.020 to about 0.125 inches, wherein said delivery slits are in an infusion segment of the tube, wherein said infusion segment includes at least one slit per inch, wherein said catheter has a cracking pressure of at least 0.1 psi, wherein said catheter does not comprise a spring, a membrane or a wick in said lumen.

14. The method according to claim 13, wherein said cracking pressure comprises at least 0.2 psi.

15. The method according to claim 13, wherein said catheter has at least two lumens and wherein each of said two lumens has an infusion segment with delivery slits, and wherein said catheter does not comprise a spring, a membrane or a wick in said two lumens.

16. The method according to claim 15, wherein said catheter has a cracking pressure of at least 0.2 psi.

17. A method for making a catheter comprising taking a catheter tube and generating a delivery slit in said catheter tube with a device selected from the group consisting of a lancet, a needle, a pointed needle, a round needle, a blade, and a beveled blade, wherein said delivery slits are in an infusion segment of the tube, wherein said infusion segment includes at least one slit per inch, wherein said catheter has a cracking pressure of at least 0.1 psi, and wherein said method does not comprise inserting a spring, a membrane or a wick in the said catheter tube.

18. The method according to claim 17, wherein said catheter has a cracking pressure of at least 0.2 psi.

19. The method according to claim 17, wherein said catheter has at least 5 delivery slits.

20. The method according to claim 17, wherein said catheter has at least 10 delivery slits.
